(19) United States
(12) Patent Application Publication
(10) Pub. No.: US 2026/0066118 A1
(43) Pub. Date: Mar. 5, 2026

(54) DATA PROCESSING DEVICE, DATA PROCESSING METHOD AND STORAGE MEDIUM

(71) Applicant: SOFTBANK GROUP CORP., Tokyo (JP)

(72) Inventor: Yoshikiyo MIZUNO, Tokyo (JP)

(73) Assignee: SOFTBANK GROUP CORP., Tokyo (JP)

(21) Appl. No.: 19/305,958

(22) Filed: Aug. 21, 2025

(30) Foreign Application Priority Data
Aug. 27, 2024 (JP) 2024-145672

(51) Int. Cl. G16H 50/20

(52) U.S. Cl. CPC G16H 50/20

(57) ABSTRACT

A data processing apparatus includes a processor configured to: acquire subject information indicating a daily state of a subject; input a first prompt to a generation model that generates information according to input data, the first prompt being a prompt for outputting a characteristic of a developmental disorder and a degree of disorder of the subject indicated by the acquired subject information; and output the characteristic of the developmental disorder and the degree of the disorder of the subject, which are output results of the generation model, to a predetermined output target.

### CROSS-REFERENCE TO RELATED APPLICATION

This application is based on and claims priority under 35 USC 119 from Japanese Patent Application No. 2024-145672 filed on Aug. 27, 2024, the disclosure of which is incorporated by reference herein.

### BACKGROUND

### Technical Field

The technique of the present disclosure relates to a data processing apparatus, a data processing method, and a storage medium of a data processing program.

### Related Art

Japanese Patent Application Laid-Open (JP-A) No. 2022-180282 discloses a persona chat bot control method executed by at least one processor, the method including steps of: receiving a user utterance; adding the user utterance to a prompt including an instruction sentence associated with a description about a character of a chat bot; encoding the prompt; and generating a chat bot utterance that responds to the user utterance, by inputting the encoded prompt into a language model.

However, in the related art, there is still room for improvement in the content output from a generation model such as the language model described above.

### SUMMARY

A data processing apparatus according to a first aspect includes a processor configured to: acquire subject information indicating a daily state of a subject; input a first prompt to a generation model that generates information according to input data, the first prompt being a prompt for outputting a characteristic of a developmental disorder and a degree of disorder of the subject indicated by the acquired subject information; and output the characteristic of the developmental disorder and the degree of the disorder of the subject, which are output results of the generation model, to a predetermined output target.

In the data processing apparatus according to the first aspect, the processor acquires the subject information indicating the daily state of the subject. In addition, the processor inputs, to the generation model, the first prompt for outputting the characteristic of the developmental disorder and the degree of the disorder of the subject indicated by the acquired subject information. The processor outputs, to the predetermined output target, the characteristic of the developmental disorder and the degree of the disorder of the subject, which are the output results of the generation model. As a result, according to the data processing apparatus, it is possible to easily ascertain the characteristic of the developmental disorder and the degree of the disorder of the subject as compared with a case of visiting a consultation destination such as a medical institution and a child consultation center to consult.

In a data processing apparatus according to a second aspect, in the first aspect, the processor is further configured to: add, to the first prompt, an instruction for outputting an observation regarding development of the subject indicated by the acquired subject information; and output, to the predetermined output target, the characteristic of the developmental disorder and the degree of the disorder of the subject, which are the output results of the generation model, and the observation regarding the development of the subject.

In the data processing apparatus according to the second aspect, the processor adds, to the first prompt, an instruction for outputting an observation regarding the development of the subject indicated by the acquired subject information. The processor outputs, to the predetermined output target, the characteristic of the developmental disorder and the degree of the disorder of the subject, which are the output results of the generation model, and an observation regarding the development of the subject. As a result, according to the data processing apparatus, it is possible to easily ascertain the observation regarding the development of the subject as compared with a case of visiting a consultation destination such as a medical institution and a child consultation center to consult.

In a data processing apparatus according to a third aspect, in the second aspect, the processor is further configured to: acquire a user input regarding at least one of course consultation in an elementary school, course consultation in a middle school, course consultation in a high school, or course consultation after graduation from a high school of the subject; add, to the first prompt, an instruction for outputting an answer to the course consultation indicated by the acquired user input; and output, to the predetermined output target, the characteristic of the developmental disorder and the degree of the disorder of the subject, which are the output results of the generation model, the observation regarding the development of the subject, and the answer regarding the course consultation.

In the data processing apparatus according to the third aspect, the processor acquires a user input regarding at least one of course consultation in an elementary school, course consultation in a middle school, course consultation in a high school, or course consultation after graduation from a high school of the subject. In addition, the processor adds, to the first prompt, an instruction for outputting an answer to the course consultation indicated by the acquired user input. The processor outputs, to the predetermined output target, the characteristic of the developmental disorder and the degree of the disorder of the subject, which are the output results of the generation model, the observation regarding the development of the subject, and the answer to the course consultation. As a result, according to the data processing apparatus, it is possible to easily ascertain a course recommended to the subject as compared with a case of visiting a consultation destination such as a medical institution and a child consultation center to consult.

In a data processing apparatus according to a fourth aspect, in any one of the first to third aspects, the processor is further configured to: acquire actual age information indicating an actual age of the subject; add, to the first prompt, an instruction for outputting a developmental age of the subject according to the actual age indicated by the acquired actual age information; and output, to the predetermined output target, the characteristic of the developmental disorder and the degree of the disorder of the subject, which are the output results of the generation model, and the developmental age according to the actual age of the subject.

In the data processing apparatus according to the fourth aspect, the processor acquires the actual age information indicating the actual age of the subject. In addition, the processor adds, to the first prompt, an instruction for outputting the developmental age of the subject according to the actual age indicated by the acquired actual age information. The processor outputs, to the predetermined output target, the characteristic of the developmental disorder and the degree of the disorder of the subject, which are the output results of the generation model, and the developmental age according to the actual age of the subject. As a result, according to the data processing apparatus, it is possible to easily ascertain the developmental age according to the actual age of the subject as compared with a case of visiting a consultation destination such as a medical institution and a child consultation center to consult.

In a data processing apparatus according to a fifth aspect, in the fourth aspect, the processor is further configured to: acquire future age information indicating a future age of the subject; add, to the first prompt, an instruction for outputting a developmental age of the subject according to the future age indicated by the acquired future age information; and output, to the predetermined output target, the characteristic of the developmental disorder and the degree of the disorder of the subject, which are the output results of the generation model, the developmental age according to the actual age of the subject, and the developmental age according to the future age of the subject.

In the data processing apparatus according to the fifth aspect, the processor acquires the future age information indicating the future age of the subject. In addition, the processor adds, to the first prompt, an instruction for outputting the developmental age of the subject according to the future age indicated by the acquired future age information. The processor outputs, to the predetermined output target, the characteristic of the developmental disorder and the degree of the disorder of the subject, which are the output results of the generation model, the developmental age according to the actual age of the subject, and the developmental age according to the future age of the subject. As a result, according to the data processing apparatus, it is possible to ascertain the future developmental age based on the current development of the subject.

In a data processing apparatus according to a sixth aspect, in any one of the first to fifth aspects, the processor is further configured to: acquire actual age information indicating an actual age of the subject; input, to the generation model, a second prompt that is a prompt for outputting the daily state of the subject to be added by next information update time according to the actual age indicated by the acquired actual age information and the characteristic of the developmental disorder and the degree of the disorder of the subject, which are output to the predetermined output target; and output the daily state of the subject to be added, which is an output result of the generation model, to the predetermined output target at a timing at which a period until the next information update time is within a predetermined number of days.

In the data processing apparatus according to the sixth aspect, the processor acquires the actual age information indicating the actual age of the subject. In addition, the processor inputs, to the generation model, a second prompt for outputting the daily state of the subject to be added by the next information update time according to the actual age indicated by the acquired actual age information, and the characteristic of the developmental disorder and the degree of the disorder of the subject, which are output to the predetermined output target. The processor outputs the daily state of the subject to be added, which is the output result of the generation model, to the predetermined output target at a timing at which the period until the next information update time is within the predetermined number of days. As a result, according to the data processing apparatus, it is possible to suppress an occurrence of a situation in which preparation of necessary information indicating the daily state of the subject is forgotten by the next information update time.

In a data processing apparatus according to a seventh aspect, in any one of the first to sixth aspects, the processor is further configured to: acquire a user input regarding a trouble about the subject; input, to the generation model, a third prompt that is a prompt for outputting an answer based on the characteristic of the developmental disorder of the subject, for the trouble indicated by the acquired user input; and output, to the predetermined output target, an answer to the trouble about the subject, which is an output result of the generation model.

In the data processing apparatus according to the seventh aspect, the processor acquires the user input regarding the trouble about the subject. In addition, the processor inputs, to the generation model, a third prompt for outputting an answer based on the characteristic of the developmental disorder of the subject for the trouble indicated by the acquired user input. The processor outputs the answer to the trouble about the subject, which is the output result of the generation model, to the predetermined output target. As a result, according to the data processing apparatus, it is possible to easily obtain advice on a trouble for the subject as compared with a case of visiting a consultation destination such as a medical institution and a child consultation center to consult.

In a data processing apparatus according to an eighth aspect, in any one of the first to seventh aspects, the processor is further configured to: acquire a user input asking about a person or facility compatible with the subject; input, to the generation model, a fourth prompt that is a prompt for outputting the person or facility compatible with the subject indicated by the acquired user input based on the characteristic of the developmental disorder and the degree of the disorder of the subject; and output, to the predetermined output target, the person or facility compatible with the subject, which is an output result of the generation model.

In the data processing apparatus according to the eighth aspect, the processor acquires the user input asking about the person or facility that is compatible with the subject. In addition, the processor inputs, to the generation model, a fourth prompt for outputting the person or facility compatible with the subject, which is indicated by the acquired user input based on the characteristic of the developmental disorder and the degree of the disorder of the subject. The processor outputs a person or facility that is compatible with the subject, which is the output result of the generation model, to the predetermined output target. As a result, according to the data processing apparatus, it is possible to support the construction of a personal relationship of the guardian of the subject and to reduce the labor of the guardian until finding a facility suitable for the subject.

A data processing method according to a ninth aspect causes a computer to execute a process including: acquiring subject information indicating a daily state of a subject; inputting a first prompt to a generation model that generates information according to input data, the first prompt being a prompt for outputting a characteristic of a developmental disorder and a degree of disorder of the subject indicated by the acquired subject information; and outputting the characteristic of the developmental disorder and the degree of the disorder of the subject, which are output results of the generation model, to a predetermined output target.

In the data processing method according to the ninth aspect, the computer executes a process of acquiring the subject information indicating the daily state of the subject. In addition, in the data processing method, the computer executes a process of inputting, to the generation model, the first prompt for outputting the characteristic of the developmental disorder and the degree of the disorder of the subject indicated by the acquired subject information. In the data processing method, the computer executes a process of outputting, to the predetermined output target, the characteristic of the developmental disorder and the degree of the disorder of the subject, which are the output results of the generation model. As a result, according to the data processing method, it is possible to easily ascertain the characteristic of the developmental disorder and the degree of the disorder of the subject as compared with a case of visiting a consultation destination such as a medical institution and a child consultation center to consult.

A data processing program stored in a non-transitory storage medium according to a tenth aspect causes a computer to execute a process including: acquiring subject information indicating a daily state of a subject; inputting a first prompt to a generation model that generates information according to input data, the first prompt being a prompt for outputting a characteristic of a developmental disorder and a degree of disorder of the subject indicated by the acquired subject information; and outputting the characteristic of the developmental disorder and the degree of the disorder of the subject, which are output results of the generation model, to a predetermined output target.

In the data processing program stored in the non-transitory storage medium according to the tenth aspect, the computer is caused to execute a process of acquiring the subject information indicating the daily state of the subject. In addition, in the data processing program, the computer is caused to execute a process of inputting, to the generation model, the first prompt for outputting the characteristic of the developmental disorder and the degree of the disorder of the subject indicated by the acquired subject information. In the data processing program, the computer is caused to execute a process of outputting, to the predetermined output target, the characteristic of the developmental disorder and the degree of the disorder of the subject, which are the output results of the generation model. As a result, according to the data processing program, it is possible to easily ascertain the characteristic of the developmental disorder and the degree of the disorder of the subject as compared with a case of visiting a consultation destination such as a medical institution and a child consultation center to consult.

### BRIEF DESCRIPTION OF THE DRAWINGS

FIG. 1 is a conceptual diagram illustrating an example of a configuration of a data processing system.

FIG. 2 is a conceptual diagram illustrating an example of main functions of a data processing apparatus and a smart device.

FIG. 3 schematically illustrates a functional configuration of a specific processing unit of the data processing apparatus.

FIG. 4 schematically illustrates an example of an operation flow of first specific processing by the data processing apparatus.

FIG. 5 is a first explanatory diagram illustrating a display example of a display.

FIG. 6 is a second explanatory diagram illustrating a display example of the display.

FIG. 7 schematically illustrates an example of an operation flow of second specific processing by the data processing apparatus.

FIG. 8 is a third explanatory diagram illustrating a display example of the display.

FIG. 9 schematically illustrates an example of an operation flow of third specific processing by the data processing apparatus.

FIG. 10 is a fourth explanatory diagram illustrating a display example of the display.

FIG. 11 schematically illustrates an example of an operation flow of fourth specific processing by the data processing apparatus.

FIG. 12 is a fifth explanatory diagram illustrating a display example of the display.

### DETAILED DESCRIPTION

Hereinafter, exemplary embodiments of a data processing apparatus, a data processing method, and a data processing program according to the technique of the disclosure will be described with reference to the accompanying drawings.

First, terms used in the following description will be described.

In the following embodiments, a processor with reference number (hereinafter, simply referred to as a “processor”) may be one arithmetic device or a combination of plural arithmetic devices. The processor may be one type of arithmetic device or a combination of plural types of arithmetic devices. Examples of the arithmetic device include a Central Processing Unit (CPU), a Graphics Processing Unit (GPU), a General-Purpose computing on Graphics Processing Units (GPGPU), and an Accelerated Processing Unit (APU).

In the following embodiments, a Random Access Memory (RAM) with reference number is a memory in which information is temporarily stored, and is used as a work memory by a processor.

In the following embodiments, a signed storage is one or more nonvolatile storage devices that store various programs, various parameters, and the like. Examples of the nonvolatile storage device include a flash memory (Solid State Drive (SSD)), a magnetic disk (for example, a hard disk), and a magnetic tape.

In the following embodiments, a signed communication I/F (Interface) is an interface including a communication processor, an antenna, and the like. The communication I/F manages communication between plural computers. Examples of the communication standard applied to the communication I/F include wireless communication standards including 5th Generation Mobile Communication System (5G), Wi-Fi®, and Bluetooth®.

In the following embodiments, “A and/or B” is synonymous with “at least one of A or B”. That is, “A and/or B” means only A, only B, or a combination of A and B. In the present specification, the same concept as “A and/or B” is applied also in a case in which three or more matters are combined and expressed by “and/or”.

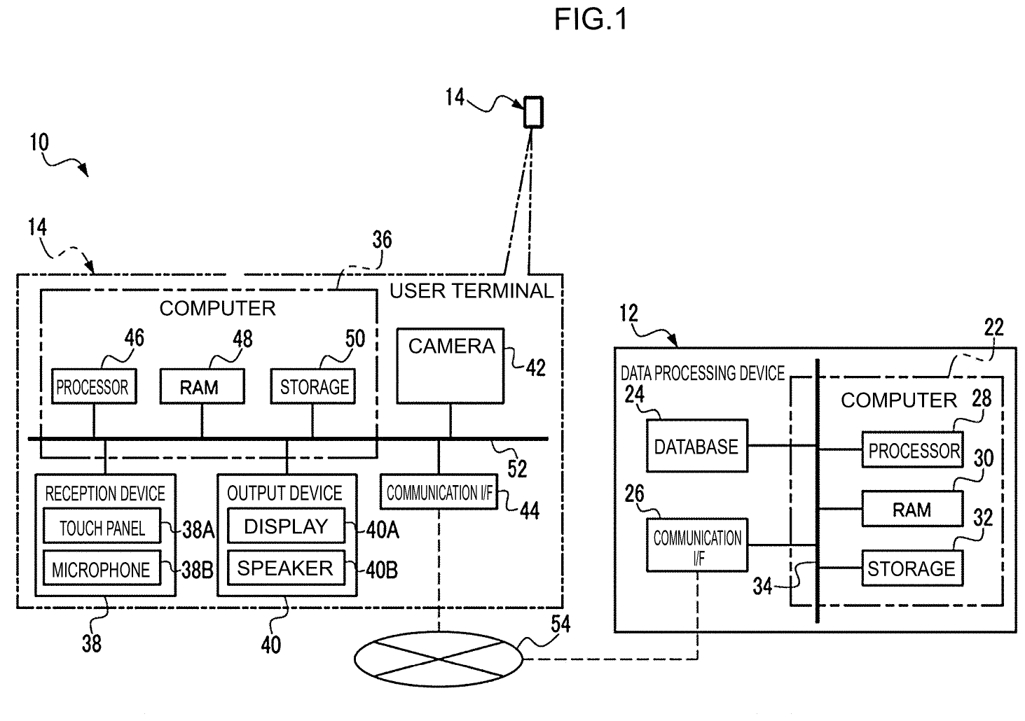

FIG. 1 illustrates an example of a configuration of a data processing system 10 according to an embodiment.

As illustrated in FIG. 1, the data processing system 10 includes a data processing apparatus 12 and a smart device 14. An example of the data processing apparatus 12 includes a server. An example of the smart device 14 includes a smartphone. In the present embodiment, the data processing apparatus 12 is an example of a “data processing apparatus” according to the technique of the disclosure, and the smart device 14 is an example of a “predetermined output target” according to the technique of the disclosure.

The data processing apparatus 12 includes a computer 22, a database 24, and a communication I/F 26. The computer 22 includes a processor 28, a RAM 30, and a storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a WAN (Wide Area Network) and/or a LAN (Local Area Network). The computer 22 is an example of a “computer” according to the technique of the disclosure, and the processor 28 is an example of a “processor” according to the technique of the disclosure.

The smart device 14 includes a computer 36, a receiving device 38, an output device 40, a camera 42, and a communication I/F 44. The computer 36 includes a processor 46, a RAM 48, and a storage 50. The processor 46, the RAM 48, and the storage 50 are connected to a bus 52. The receiving device 38, the output device 40, and the camera 42 are also connected to the bus 52.

The receiving device 38 includes a touch panel 38A, a microphone 38B, and the like, and receives a user input. The touch panel 38A detects contact of a pointer (for example, a pen, a finger, or the like) and receives a user input by the contact of the pointer. The microphone 38B receives a user input by voice by detecting the voice of the user. A control unit 46A transmits data indicating the user input received by the touch panel 38A and the microphone 38B to the data processing apparatus 12. In the data processing apparatus 12, a specific processing unit 290 acquires data indicating a user input.

The output device 40 includes a display 40A, a speaker 40B, and the like, and presents data to a person by outputting the data in an expression (for example, voice and/or text) perceivable by a human. The display 40A displays visible information such as text and images in accordance with an instruction from the processor 46. The speaker 40B outputs a voice in accordance with an instruction from the processor 46. The camera 42 is a small digital camera on which an optical system such as a lens, a diaphragm, and a shutter and an imaging element such as a Complementary Metal-Oxide-Semiconductor (CMOS) image sensor or a Charge Coupled Device (CCD) image sensor are mounted.

The communication I/F 44 is connected to the network 54. The communication I/Fs 44 and 26 manage exchange of various types of information between the processor 46 and the processor 28 via the network 54.

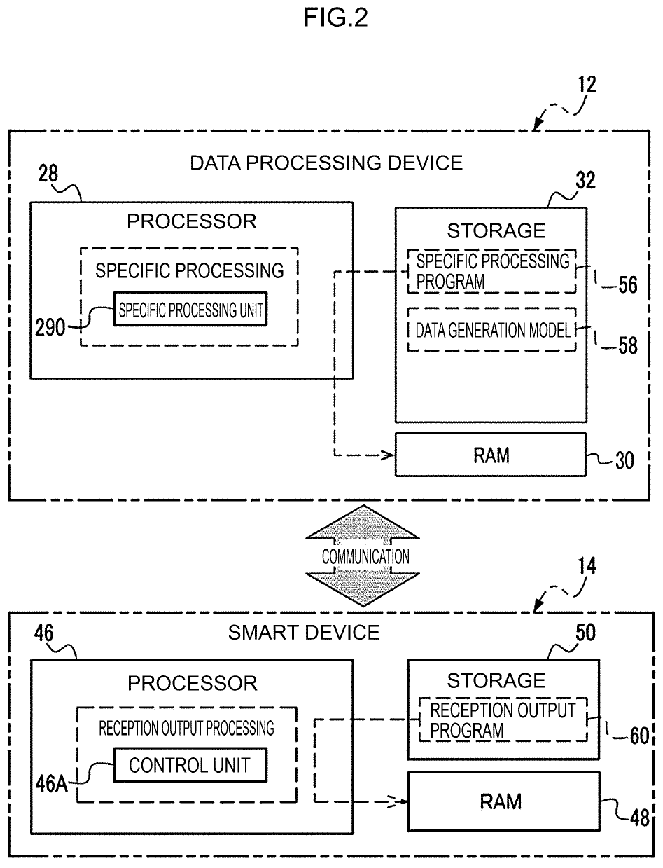

FIG. 2 illustrates an example of main functions of the data processing apparatus 12 and the smart device 14.

As illustrated in FIG. 2, in the data processing apparatus 12, specific processing is performed by the processor 28. The storage 32 stores a specific processing program 56. The specific processing program 56 is an example of a “data processing program” according to the technique of the disclosure. The “data processing program” according to the technique of the disclosure may also be implemented as a program product. The processor 28 readouts the specific processing program 56 from the storage 32 and executes the read specific processing program 56 on the RAM 30. The specific processing is realized by the processor 28 operating as the specific processing unit 290 in accordance with the specific processing program 56 executed on the RAM 30.

The storage 32 stores a data generation model 58. The data generation model 58 is used by the specific processing unit 290. The data generation model 58 is an example of a “generation model” according to the technique of the disclosure.

In the smart device 14, reception output processing is performed by the processor 46. The storage 50 stores a reception output program 60. The reception output program 60 is used in combination with the specific processing program 56 by the data processing system 10. The processor 46 readouts the reception output program 60 from the storage 50 and executes the read reception output program 60 on the RAM 48. The reception output processing is realized by the processor 46 operating as the control unit 46A in accordance with the reception output program 60 executed on the RAM 48.

Next, a functional configuration of the specific processing unit 290 will be described.

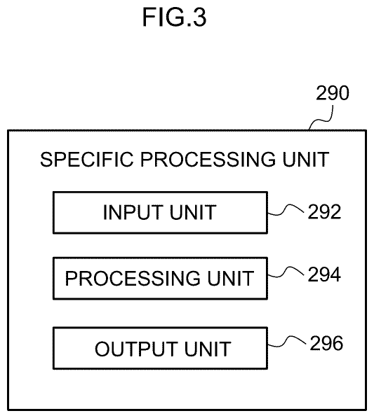

FIG. 3 is a block diagram illustrating an example of the functional configuration of the specific processing unit 290.

As illustrated in FIG. 3, the specific processing unit 290 includes an input unit 292, a processing unit 294, and an output unit 296.

The input unit 292 acquires a user input received by the smart device 14. Specifically, the input unit 292 acquires data of at least one of text, voice, image, or a moving image of the user received by the smart device 14.

The processing unit 294 performs specific processing using the data generation model 58. Specifically, the processing unit 294 inputs a predetermined prompt to the data generation model 58 and obtains an output result.

The output unit 296 transmits (outputs) the output result of the data generation model 58, that is, the result of the specific processing, to the smart device 14. In the smart device 14, the control unit 46A causes the output device 40 to output the result of the specific processing.

The data generation model 58 is a so-called generation-system AI (Artificial Intelligence). Examples of the data generation model 58 include generation-system AI such as ChatGPT (Internet search <URL:https//openai.com/blog/chatgpt>) and Gemini (Internet search <URL:https://gemini.google.com/?hl=ja>). The data generation model 58 is obtained by causing a neural network to perform deep learning. To the data generation model 58, a prompt including an instruction is input, and inference data such as voice data indicating voice, text data indicating text, image data indicating an image, and moving image data indicating a moving image is also input. The data generation model 58 performs inferring based on the input inference data in accordance with the instruction indicated by the prompt, and outputs the inference result in a data format such as voice data, text data, graph data, table data, and image data. Here, the inference refers to, for example, analysis, classification, prediction, and/or summary.

Next, the operation of the data processing system 10 will be described.

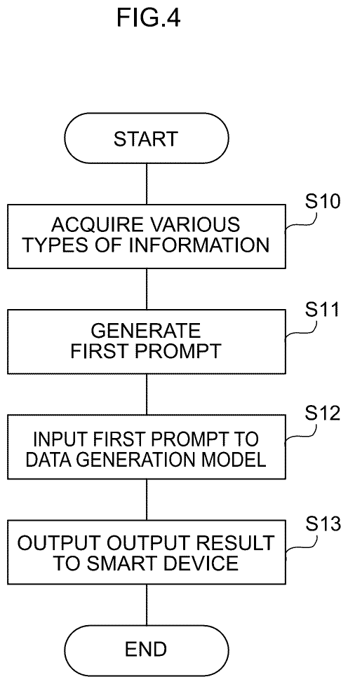

First, an example of a flow of first specific processing in the specific processing will be described with reference to FIG. 4. For example, the first specific processing is performed in a case in which the data processing apparatus 12 acquires a user input for designating a subject in a predetermined application received by the smart device 14. The subject is, for example, a human including a child and an adult suspected of having a developmental disorder. The embodiment is not limited thereto, and the human who has been diagnosed as having a developmental disorder by a doctor or the like may be referred to as the “subject”. Here, the flow of the first specific processing illustrated in FIG. 4 is an example of a “data processing method” according to the technique of the disclosure.

In Step S10 illustrated in FIG. 4, the processor 28 acquires various types of information. Then, the processor 28 proceeds to Step S11. Here, the various types of information include at least registration information indicating the name, gender, and the like of the subject and subject information indicating the daily state or daily behavior of the subject.

The registration information is information of the subject registered in advance in the application by a guardian (for example, a parent) of the subject. For example, the registration information includes a face picture, a name, a gender, a date of birth, an address, favorite things, and the like of the subject. The registration information registered by the guardian is stored in the storage 32 in association with the subject. In Step S10, the processor 28 may acquire the registration information corresponding to the subject designated in the user input from the storage 32 as various types of information. The date of birth of the subject included in the registration information is an example of “actual age information indicating the actual age of the subject” according to the technique of the disclosure.

The subject information includes, for example, image data or moving image data indicating a state of an action such as hand play, motion, reading and writing, conversation, meal, action on a task imposed on the subject, and tantrums as the daily state of the subject, and image data indicating an evaluation of a task imposed on the subject and a state of the subject in a developmental support facility. In the present embodiment, partial data of image data and moving image data stored in the storage 50 of the smart device 14 owned by the guardian is the subject information. For example, the subject information may be image data and moving image data in which the subject close to the actual age calculated from the registration information (for example, within the last one year) is captured, and the image data and the moving image data being extracted by performing known image processing on the image data and the moving image data stored in the storage 50 by the processor 46. The subject information may be image data and moving image data selected by the guardian from the image data and moving image data stored in the storage 50. The processor 46 transmits the determined subject information to the data processing apparatus 12. In Step S10, the processor 28 may acquire the subject information transmitted from the processor 46 as the various types of information.

Here, in a case in which a predetermined user input is made by the guardian, the processor 28 acquires information based on the predetermined user input as the various types of information in Step S10. As the predetermined user input, the guardian may perform course consultation of the subject and question the future developmental age of the subject using the smart device 14. In a case in which a request for course consultation of the subject is received as the predetermined user input, the processor 46 transmits consultation information regarding the course consultation, and in a case in which a question about the future developmental age of the subject is received as the predetermined user input, the processor transmits future age information indicating the future age of the subject to the data processing apparatus 12. In Step S10, the processor 28 may acquire the consultation information and the future age information transmitted from the processor 46 as the various types of information.

In Step S11, the processor 28 generates a first prompt for outputting a diagnosis result on the development of the subject based on the various types of information acquired in Step S10. Then, the processor 28 proceeds to Step S12.

Here, the processor 28 adds, to the first prompt, at least an instruction sentence for including a face picture, a name, a gender, a date of birth, a characteristic of a developmental disorder, a degree of the disorder, an observation regarding development, and a developmental age according to an actual age of the subject in the above diagnosis result. In a case in which the predetermined user input is made by the guardian, the processor 28 adds, to the first prompt, an instruction sentence for including at least one of an answer to the course consultation of the subject or the developmental age according to the future age in the diagnosis result.

For example, in a case in which the registration information, the subject information, the future age information, and the consultation information are included in the various types of information acquired in Step S10, the processor 28 generates the first prompt as follows.

### “First Prompt”

Please output the results of diagnosis on the subject's development indicated by the following registration information and subject information.

Registration information-->face picture.jpeg, name: user A, gender: boy, date of birth: Dec. 17, 2020

Subject information-->image data 1.jpeg, image data 2.jpeg, moving image data 1.mp4, and moving image data 2.mp4.

Please include the following in the diagnosis result.
(1) Face picture of subject
(2) Name of subject
(3) Gender of subject
(4) Date of birth of subject
(5) Characteristic of developmental disorder of subject
(6) Degree of disorder of subject
(7) Observation regarding development of subject
(8) Developmental age according to actual age of subject
(9) Developmental age according to future age of subject
(10) Answer to the course consultation of subject
-->In (8), please output a radar chart showing the developmental age of each of six items including speech, interpersonal relationship, movement motion, hand motion, basic habit, and language understanding, and scores according to each developmental age; and two types of comprehensive developmental ages based on the six items of developmental ages.
22 Please set the future age in (9) “20 years and 0 months” indicated by the obtained future age information.
-->Please output the answer to (10) in consideration of the consultation contents such as “how about the course of an elementary school?” indicated in the obtained consultation information.

In Step S12, the processor 28 inputs the first prompt generated in Step S11 to the data generation model 58, and acquires the output result by the data generation model 58. Then, the processor 28 proceeds to Step S13.

In Step S13, the processor 28 outputs the output result of the data generation model 58 in Step S12 to the smart device 14 of a guardian (hereinafter, “guardian A”) of a user A who is the subject. In the smart device 14, the processor 46 causes the output device 40 to output the result of the first specific processing. As a result, a diagnosis result regarding development of the user A is displayed on the display 40A. The diagnosis result is stored in the storage 32 in association with the user A. Then, the processor 28 ends the first specific processing.

Next, a display example displayed on the display 40A of the smart device 14 of the guardian A based on the execution of the first specific processing will be described.

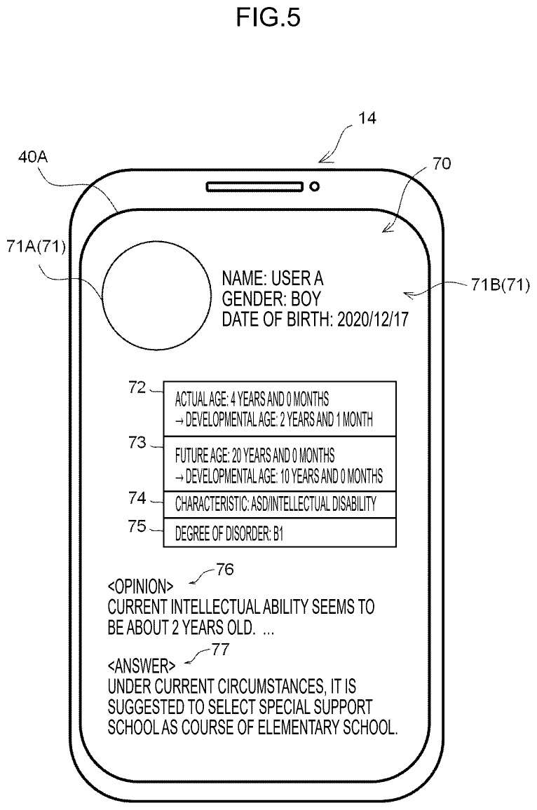

FIG. 5 is a first explanatory diagram illustrating a display example of the display 40A. For example, FIG. 5 illustrates a result display screen 70 showing a diagnosis result regarding the development of the user A.

The result display screen 70 illustrated in FIG. 5 is provided with a registration information display portion 71, a result display portion 72, a result display portion 73, a result display portion 74, a result display portion 75, a message display portion 76, and a message display portion 77.

The registration information display portion 71 is a part that displays the registration information of the user A, and includes a first display portion 71A that displays a face picture and a second display portion 71B that displays the name, the gender, and the date of birth. In FIG. 5, for convenience of description, the inside of the first display portion 71A is blank, but actually, the face picture of the user A indicated by the image data (for example, face picture.jpeg) included in the first prompt is displayed. “Name: user A, gender: boy date of birth: 2020 Dec. 17” is displayed on the second display portion 71B, and the contents of the registration information included in the first prompt are displayed.

The result display portion 72 is a part that displays the developmental age corresponding to the actual age of the user A. The developmental age is an index indicating the age of the current development of the user A according to the actual age. “Actual age: 4 years and 0 months-->developmental age: 2 years and 1 month” is displayed on the result display portion 72, and it is shown that the developmental age according to the actual age of the user A is 2 years and 1 month.

The result display portion 73 is a part that displays the developmental age according to the future age (for example, 20 years old and 0 months) designated by the first prompt of the user A. The developmental age is an index indicating the age of the future development of the user A according to the future age designated in the first prompt. “Future age: 20 years and 0 months-->developmental age: 10 years and 0 months” is displayed on the result display portion 73, and it is shown that the developmental age when the user A becomes 20 years and 0 months is 10 years and 0 months.

The result display portion 74 is a part that displays the characteristic of the developmental disorder of the user A. “Characteristic: ASD/intellectual disability” is displayed on the result display portion 74, and it is shown that the user A is autism spectrum disorder (ASD) and also has intellectual disability. The characteristic of the developmental disorder that may be displayed on the result display portion 74 includes known characteristics such as attention deficit hyperactivity disorder (ADHD), learning disorder (LD), language development delay, and developmental coordination disorder (DCD) in addition to ASD.

The result display portion 75 is a part that displays the degree of the disorder of the user A. “Degree of disorder: B1” is displayed on the result display portion 75, and it is shown that the user A has a moderate intellectual disability with the grade of the rehabilitation notebook being “B1”. The degrees of disorder that may be displayed on the result display portion 75 include, in addition to B1, known degrees such as “A1” that is severe intellectual disability,“A2” that is moderate intellectual disability and also combines a physical disorder of Grade 3 or higher, and “B2” that is mild intellectual disability.

The message display portion 76 is a part that displays an observation regarding development of the user A as a message to the guardian A. “The current intellectual ability seems to be about 2 years old . . . . ” is displayed on the message display portion 76, and an observation regarding development of the user A as if a doctor has expressed an observation is shown.

The message display portion 77 is a part that displays an answer to the course consultation designated by the first prompt of the user A as a message to the guardian A. “Under the current circumstances, it is suggested to select a special support school as the course of an elementary school.” is displayed on the message display portion 77, and a proposal for the course of an elementary school of the user A is shown. The answer regarding the course consultation that may be displayed on the message display portion 77 is not limited to the answer regarding the course of the elementary school, and includes an answer regarding at least one of the course consultation in the elementary school, the course consultation in the middle school, the course consultation in the high school, and the course consultation after graduation from the high school, which is designated by the first prompt.

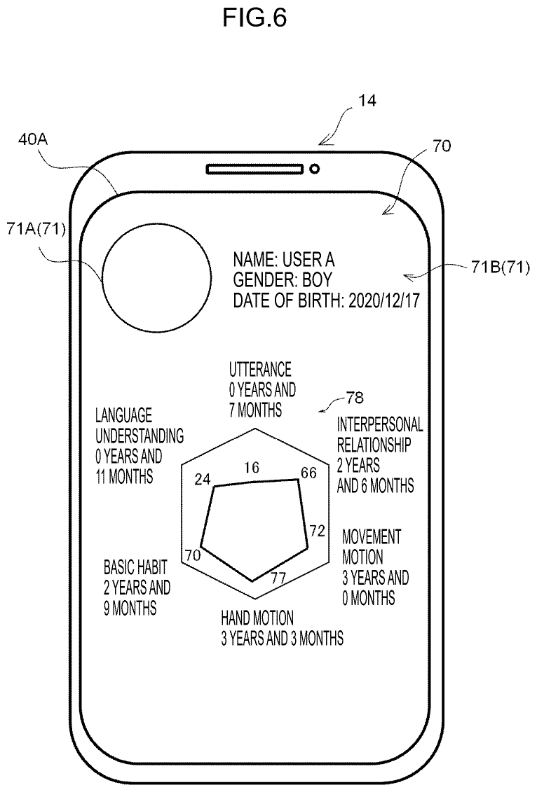

FIG. 6 is a second explanatory diagram illustrating a display example of the display 40A. For example, FIG. 6 illustrates a result display screen 70 in which the display content is switched by an operation on the smart device 14 by the guardian A.

The result display screen 70 illustrated in FIG. 6 is provided with a registration information display portion 71 and a chart display portion 78. The display content of the registration information display portion 71 is similar to that in FIG. 5, and thus description thereof will be omitted.

The chart display portion 78 is a part that displays a radar chart indicating the developmental age of each of the plural items designated by the first prompt of the user A and the score according to each developmental age. The radar chart illustrated in FIG. 6 shows the developmental age of each of the six items of speech, interpersonal relationship, movement motion, hand motion, basic habit, and language understanding, and the score according to each developmental age. For example, according to the radar chart, it is possible to ascertain that the developmental age for the utterance item of the user A is 0 years and 7 months, and the score according to “developmental age: 0 years and 7 months” is 16 points out of 100 points.

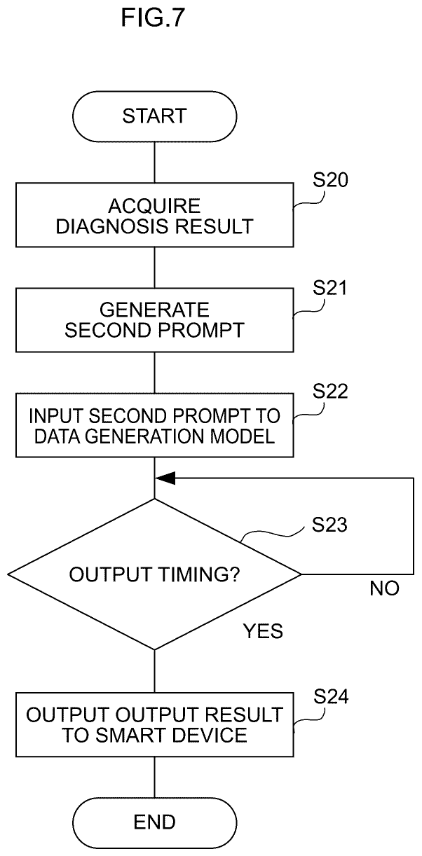

Next, an example of a flow of the second specific processing in the specific processing will be described with reference to FIG. 7. For example, the second specific processing is started at a predetermined timing after the execution of the first specific processing.

In Step S20 illustrated in FIG. 7, the processor 28 acquires the diagnosis result regarding the development of the subject stored in the storage 32. In the following description, it is assumed that the processor 28 acquires the diagnosis result corresponding to the user A as the subject from the storage 32. Then, the processor 28 proceeds to Step S21.

In Step S21, the processor 28 generates a second prompt for outputting the daily state of the user A to be added by the next information update time based on the diagnosis result acquired in Step S20. The daily state of the user A to be added is, for example, a state of an action in a specific situation, environment, or the like of the user A. Then, the processor 28 proceeds to Step S22.

Here, in the data processing system 10 according to the present embodiment, the processor 28 periodically (for example, once every half year, once a year or the like) updates the diagnosis result regarding the development of the subject. In a case in which the update time comes, the processor 28 notifies the smart device 14 of the guardian corresponding to the subject as an update target that the update is to be performed. Thereafter, the processor 28 acquires the diagnosis result corresponding to the subject as the update target from the storage 32 based on a response operation performed by the guardian. Based on the acquired diagnosis result, the processor 28 executes the first specific processing illustrated in FIG. 4 again and generates a new diagnosis result.

For example, the processor 28 generates a second prompt as follows: In the following second prompt, for convenience of description, only the date of birth, the characteristic of the developmental disorder, and the degree of the disorder included in the diagnosis result are illustrated, but actually, the entire contents of the diagnosis result stored in the storage 32 are included.

### “Second Prompt”

Please output the daily state of the user A to be added by the next information update time (X month X day, 20XX) based on the following diagnosis result regarding the development of the user A.
Diagnosis result
->Date of birth: Dec. 17, 2020
->Characteristic of developmental disorder: ASD/Intellectual disability
->Degree of disorder: B1

In Step S22, the processor 28 inputs the second prompt generated in Step S21 to the data generation model 58, and acquires the output result by the data generation model 58. Then, the processor 28 proceeds to Step S23.

In Step S23, the processor 28 determines whether or not an output timing to output the result of the second specific processing to the smart device 14 of the guardian A has arrived. Here, in a case in which it is determines that the output timing has arrived (Step S23: YES), the processor 28 proceeds to Step S24. In a case in which it is determined that the output timing has not arrived (Step S23: NO), the processor 28 waits until the output timing arrives. For example, the processor 28 determines that the output timing has arrived in a case in which the period until the next information update time is within a predetermined number of days.

In Step S24, the processor 28 outputs the output result of the data generation model 58 in Step S22 to the smart device 14 of the guardian A. In the smart device 14, the processor 46 causes the output device 40 to output the result of the second specific processing. As a result, the daily state of the user A to be added by the next information update time is displayed on the display 40A. Then, the processor 28 ends the second specific processing.

Next, a display example displayed on the display 40A of the smart device 14 of the guardian A based on the execution of the second specific processing will be described.

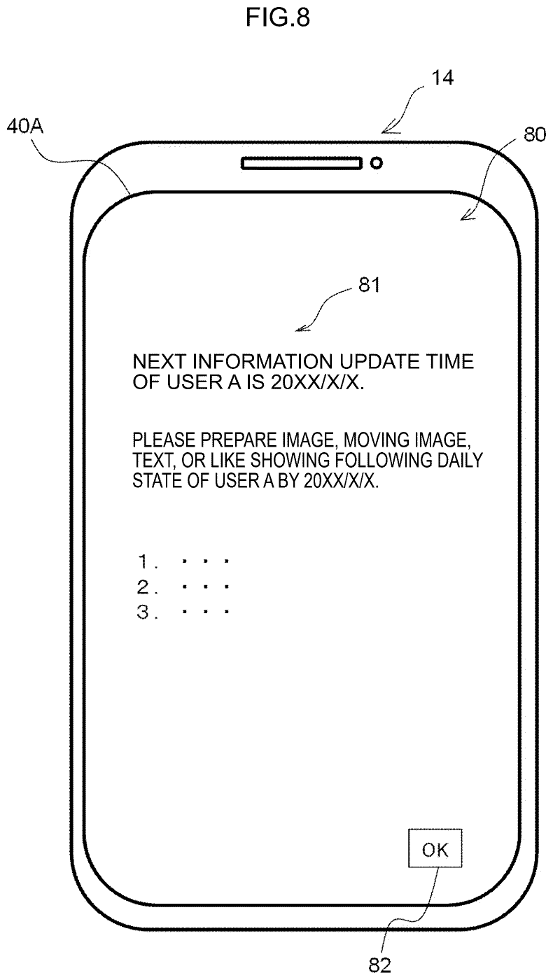

FIG. 8 is a third explanatory diagram illustrating a display example of the display 40A. For example, FIG. 8 illustrates a notification screen 80 for notifying the guardian A of the daily state of the user A to be added by the next information update time.

The notification screen 80 illustrated in FIG. 8 is provided with a message display portion 81 and an OK button 82.

The message display portion 81 is a part that displays the daily state of the user A to be added as a message for the guardian A. For example, the message display portion 81 displays that “the next information update time of the user A is 20XX/X/X. Please prepare an image, a moving image, text, or the like showing the following daily state of the user A by 20XX/X/X. 1 . . . 2 . . . 3 . . . ”, and shows the daily state of the user A to be prepared by the guardian A.

The OK button 82 is a button for changing the display content of the display 40A to a predetermined home screen (not illustrated). In a case in which the OK button 82 is operated, the processor 46 changes the display content of the display 40A from the notification screen 80 to the home screen.

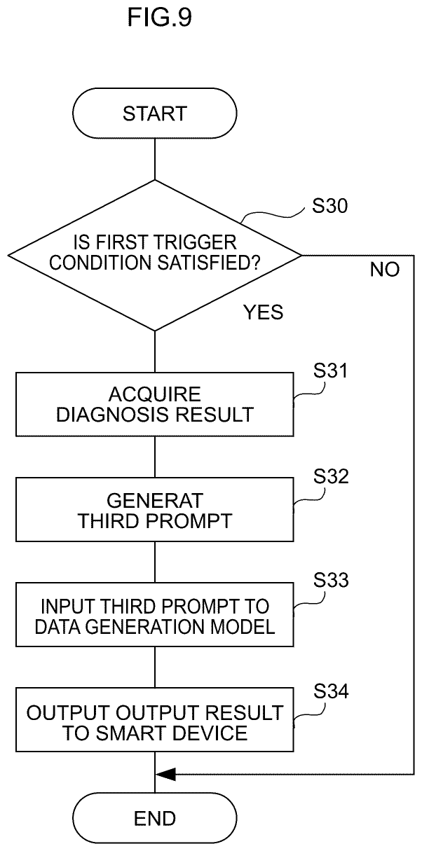

Next, an example of a flow of third specific processing in the specific processing will be described with reference to FIG. 9. For example, the third specific processing is repeatedly and automatically executed every lapse of a certain period after the execution of the first specific processing.

In Step S30 illustrated in FIG. 9, the processor 28 determines whether a first trigger condition is satisfied. Here, in a case in which it is determines that the first trigger condition is satisfied (Step S30: YES), the processor 28 proceeds to Step S31. In a case in which it is determined that the first trigger condition is not satisfied (Step S30: NO), the processor 28 ends the third specific processing. The processor 28 acquires a user input received by the smart device 14, and executes known natural language processing on the text of the user input. In a case in which it is specified that the user input relates to the trouble for the subject as a result of analyzing the context of the text, the processor 28 determines that the first trigger condition is satisfied. Hereinafter, a description will be made on the assumption that the guardian A has made a user input regarding a trouble for the user A.

In Step S31, the processor 28 acquires the diagnosis result regarding the development of the user A stored in the storage 32. Then, the processor 28 proceeds to Step S32.

In Step S32, the processor 28 generates a third prompt for outputting an answer based on the characteristic of the developmental disorder of the user A regarding the trouble indicated by the user input based on the diagnosis result acquired in Step S31. Then, the processor 28 proceeds to Step S33.

Here, the processor 28 adds, to the third prompt, at least an instruction sentence for outputting plural options including an optimal solution based on the characteristic of the developmental disorder of the subject. For example, the processor 28 generates a third prompt as follows: In the following third prompt, for convenience of description, only the characteristic of the developmental disorder included in the diagnosis result is illustrated, but actually, the entire contents of the diagnosis result stored in the storage 32 are included.

### “Third Prompt”

Troubles regarding the user A input by the guardian A are as follows.
->The user A does not eat udon.

Please output an answer to the above trouble based on the following diagnosis result regarding development of the user A. For the answer, please output plural options including the optimal solution based on the characteristic of the developmental disorder of the user A.

### Diagnosis Result

->Characteristic of developmental disorder: ASD/Intellectual disability

In Step S33, the processor 28 inputs the third prompt generated in Step S32 to the data generation model 58, and acquires the output result by the data generation model 58. Then, the processor 28 proceeds to Step S34.

In Step S34, the processor 28 outputs the output result of the data generation model 58 in Step S33 to the smart device 14 of the guardian A. In the smart device 14, the processor 46 causes the output device 40 to output the result of the third specific processing. As a result, an answer to the trouble for the user A is displayed on the display 40A. Then, the processor 28 ends the third specific processing.

Next, a display example displayed on the display 40A of the smart device 14 of the guardian A based on the execution of the third specific processing will be described.

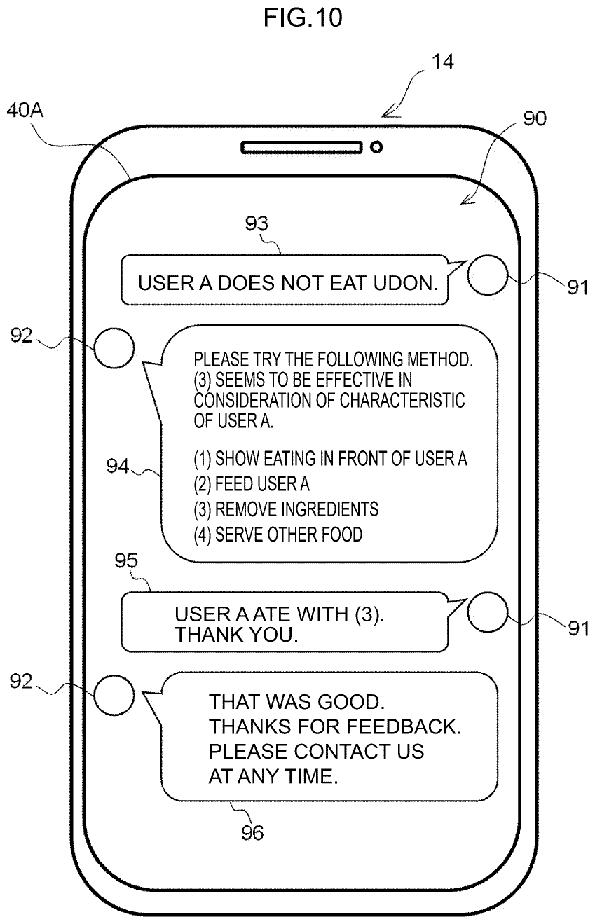

FIG. 10 is a fourth explanatory diagram illustrating a display example of the display 40A. For example, FIG. 10 illustrates a talk screen 90 illustrating exchange of messages between the guardian A and an AI agent.

On the talk screen 90 illustrated in FIG. 10, a guardian-side icon 91, an AI-side icon 92, a message 93, a message 94, a message 95, and a message 96 are displayed.

The guardian-side icon 91 is an icon indicating the guardian A on the talk screen 90.

The AI-side icon 92 is an icon indicating the AI agent on the talk screen 90.

The message 93 indicates a message “the user A does not eat udon”, received as the text input by the guardian A.

The message 94 indicates the answer output from the data generation model 58 to the trouble indicated by the message 93, that is, the result of the third specific processing. For example, the answer indicated by the message 94 is “please try the following method. (3) seems to be effective in consideration of the characteristic of the user A. (1) Show eating in front of user A, (2) Feed user A, (3) Remove the ingredients, (4) Serve other food”. As described above, the message 94 indicates plural options including the optimal solution based on the characteristic of the developmental disorder of the user A.

The message 95 indicates a message that “User A ate with (3). Thank you”, received as the text input by the guardian A, as a response to the message 94.

The message 96 indicates an answer output by the data generation model 58 in response to the message indicated by the message 95. For example, the answer indicated by the message 96 is “That was good. Thank you for feedback. Please contact us at any time”.

Here, the processor 28 may fine-tune the data generation model 58 by using the exchange of messages between the guardian A and the AI agent, which are indicated as the message 93, the message 94, and the message 95, as learning data. In this case, the processor 28 performs fine tuning of the data generation model 58 by appropriately using a known method disclosed in, for example, Internet search <URL:https://www.brainpad.co.jp/doors/knowledge/01_chatgpt_fine_tuning_internal_documents/>, Internet search <URL: https://di-acc2.com/programming/python/25924/>, or the like.

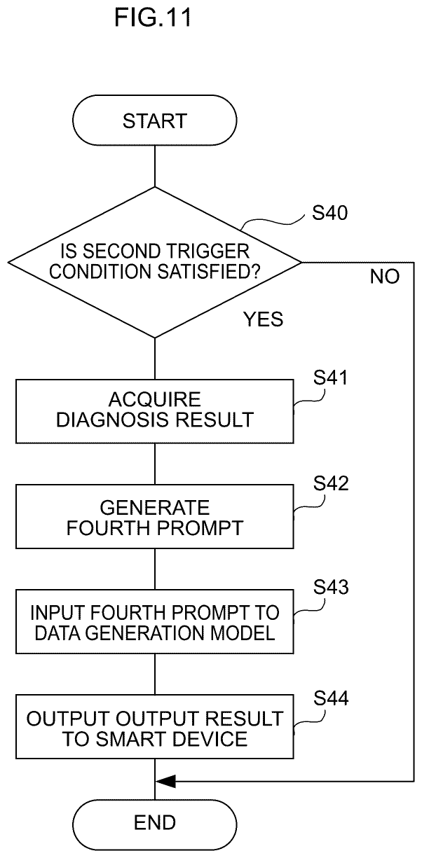

Next, an example of a flow of fourth specific processing in the specific processing will be described with reference to FIG. 11. For example, the fourth specific processing is repeatedly and automatically executed every lapse of a certain period after the execution of the first specific processing.

In Step S40 illustrated in FIG. 11, the processor 28 determines whether a second trigger condition is satisfied. Here, in a case in which it is determined that the second trigger condition is satisfied (Step S40: YES), the processor 28 proceeds to Step S41. In a case in which it is determined that the second trigger condition is not satisfied (Step S40: NO), the processor 28 ends the fourth specific processing. The processor 28 acquires a user input received by the smart device 14, and executes known natural language processing on the text of the user input. In a case in which the processor 28 identifies that the user input is asking about a person or facility compatible with the subject as a result of analyzing the context of the text, the processor determines that the second trigger condition is satisfied. The person who is compatible with the subject is another user different from the subject registered in the application. The facility that is compatible with the subject includes a developmental support facility and a medical institution. In the following description, it is assumed that the guardian A has made a user input asking about another user who is compatible with the user A.

In Step S41, the processor 28 acquires the diagnosis result regarding the development of the user A stored in the storage 32. Then, the processor 28 proceeds to Step S42.

In Step S42, the processor 28 generates a fourth prompt for outputting another user who is compatible with the user A, which is indicated by the user input, based on the diagnosis result acquired in Step S41. Then, the processor 28 proceeds to Step S43.

For example, the processor 28 generates a fourth prompt as follows: In the following fourth prompt, for convenience of description, only the characteristic of the developmental disorder, and the degree of the disorder included in the diagnosis result are illustrated, but actually, the entire contents of the diagnosis result stored in the storage 32 are included.

### “Fourth Prompt”

Please output the top two other users who are compatible with the user A based on the following diagnosis result regarding development of the user A.

Diagnosis result
->Characteristic of developmental disorder: ASD/Intellectual disability
->Degree of disorder: B1

Please also include the following in the output contents.
(1) Face pictures of other users
(2) Doughnut graph showing compatibility (%) between the user A and other users
(3) Names and genders of other users
(4) Residential areas of other users (municipality)
(5) Characteristics of developmental disorders of other users
(6) Generations of other users
(7) Favorite things of other users

In Step S43, the processor 28 inputs the fourth prompt generated in Step S42 to the data generation model 58, and acquires the output result by the data generation model 58. Then, the processor 28 proceeds to Step S44.

In Step S44, the processor 28 outputs the output result of the data generation model 58 in Step S43 to the smart device 14 of the guardian A. In the smart device 14, the processor 46 causes the output device 40 to output the result of the fourth specific processing. As a result, another user who is compatible with the user A is displayed on the display 40A. Then, the processor 28 ends the fourth specific processing.

Next, a display example displayed on the display 40A of the smart device 14 of the guardian A based on the execution of the fourth specific processing will be described.

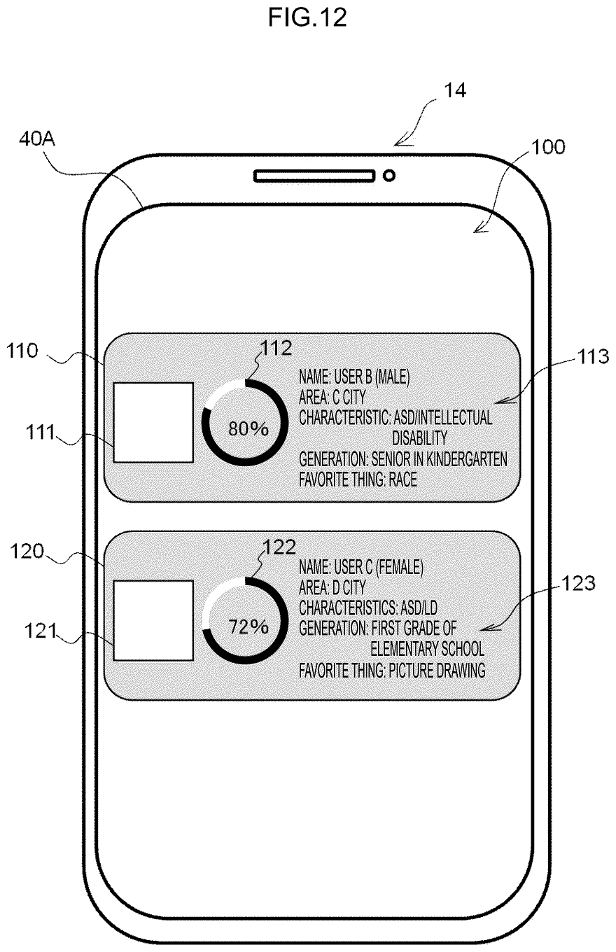

FIG. 12 is a fifth explanatory diagram illustrating a display example of the display 40A. For example, FIG. 12 illustrates a user display screen 100 showing another user who is compatible with the user A.

The user display screen 100 illustrated in FIG. 12 displays first user information 110 that is information regarding a user B who is another user and is a first user having a compatibility with the user A, and second user information 120 that is information regarding a user C who is another user and is a second user having a compatibility with the user A.

The first user information 110 includes image information 111, graph information 112, and text information 113.

The image information 111 indicates a face picture of the user B registered as registration information of the user B in the application. In FIG. 12, for convenience of description, the inside of the image information 111 is blank, but the face picture of the user B is actually displayed.

In the graph information 112, a doughnut graph indicating compatibility between the user A and the user B is indicated. According to the doughnut graph, it is possible to ascertain that the compatibility between the user A and the user B is 80%.

In the text information 113, text that introduces the user B is indicated. The text includes the name, the gender, the residential area, the characteristic of the developmental disorder, the generation, and the favorite thing of the user B designated in the fourth prompt. Specifically, the text information 113 indicates text of “name: user B (male) area: C city characteristic: ASD/intellectual disability generation: senior favorite things: race”.

The second user information 120 includes image information 121, graph information 122, and text information 123.

The image information 121 indicates a face picture of the user C registered as registration information of the user C in the application. In FIG. 12, for convenience of description, the inside of the image information 121 is blank, but the face picture of the user C is actually displayed.

In the graph information 122, a doughnut graph indicating compatibility between the user A and the user C is indicated. According to the doughnut graph, it is possible to ascertain that the compatibility between the user A and the user C is 72%.

In the text information 123, text that introduces the user C is indicated. The text includes the name, the gender, the residential area, the characteristic of the developmental disorder, the generation, and the favorite thing of the user C designated in the fourth prompt. Specifically, the text information 123 indicates text of “name: user C (female) area: D city characteristic: ASD/LD generation: first year of elementary school favorite thing: Picture drawing”.

Here, the application is provided with a message function capable of exchanging messages between users. For example, the guardian A who looks at the user display screen 100 illustrated in FIG. 12 may establish a personal relationship between the guardians by transmitting a message to the guardian of the user B or the user C who is compatible with the user A.

As described above, in the data processing apparatus 12, the processor 28 acquires various types of information including the subject information indicating the daily state of the subject. The processor 28 inputs, to the data generation model 58, the first prompt for outputting a diagnosis result regarding the development of the subject indicated by the acquired various types of information. The processor 28 outputs, to the smart device 14, a diagnosis result regarding the development of the subject, which is an output result of the data generation model 58. As a result, according to the data processing apparatus 12, it is possible to easily ascertain the diagnosis result regarding the development of the subject as compared with a case of visiting a consultation destination such as a medical institution and a child consultation center to consult. The diagnosis result includes, for example, the characteristic of developmental disorder, the degree of the disorder, an observation regarding the development of the subject, the developmental age according to the actual age, the future developmental age based on the current development, a course recommended to the subject, and the like.

In addition, in the data processing apparatus 12, the processor 28 inputs the second prompt for outputting the daily state of the subject to be added by the next information update time to the data generation model 58 according to the acquired diagnosis result regarding the development of the subject. The processor 28 outputs the daily state of the subject to be added, which is the output result of the data generation model 58, to the smart device 14 at a timing at which the period until the next information update time is within the predetermined number of days. As a result, according to the data processing apparatus 12, it is possible to reduce an occurrence of a situation in which the guardian forgets preparation of necessary information indicating the daily state of the subject by the next information update time.

In the data processing apparatus 12, the processor 28 acquires the user input regarding the trouble about the subject. The processor 28 inputs, to the data generation model 58, the third prompt for outputting an answer based on the characteristic of the developmental disorder of the subject for the trouble indicated by the acquired user input. The processor 28 outputs, to the smart device 14, an answer to the trouble about the subject which is the output result of the data generation model 58. As a result, according to the data processing apparatus 12, it is possible to easily obtain advice on a trouble for the subject as compared with a case of visiting a consultation destination such as a medical institution and a child consultation center to consult.

In the data processing apparatus 12, the processor 28 acquires the user input asking about the person or facility that is compatible with the subject. The processor 28 inputs, to the data generation model 58, the fourth prompt for outputting a person or facility compatible with the subject, which is indicated by the acquired user input based on the diagnosis result regarding the development of the subject. The processor 28 outputs the person or facility that is compatible with the subject, which is the output result of the data generation model 58, to the smart device 14. As a result, according to the data processing apparatus 12, it is possible to support the construction of a personal relationship of the guardian of the subject and to reduce the labor of the guardian until finding a facility suitable for the subject.

### (Other Features)

Although the functions of the data processing apparatus 12 have been mainly described above as the data processing system 10, the data processing system 10 may not necessarily be implemented in a server. The data processing system 10 may be implemented as a general information processing system. The disclosure may be implemented as, for example, a software program operating on a personal computer or an application operating on a smartphone or the like. The method according to the disclosure may be provided to a user in a Software as a Service (SaaS) format.

In the above embodiment, the smart device 14 has been described as an example of the “predetermined output target” according to the technique of the disclosure, but the disclosure is not limited thereto. For example, an electronic document in which an individual support plan is digitized may be an example of the “predetermined output target” according to the technique of the disclosure. In this case, the diagnosis result regarding the development of the subject, which is the result of the first specific processing, is output to the electronic document.

In the above embodiment, the embodiment in which the specific processing is performed by one computer 22 has been described, but the technique of the disclosure is not limited thereto, and distributed processing for the specific processing by plural computers including the computer 22 may be performed.

In the above embodiment, the description has been made by exemplifying the embodiment in which the specific processing program 56 is stored in the storage 32, but the technique of the disclosure is not limited thereto. For example, the specific processing program 56 may be stored in a portable computer-readable non-transitory storage medium such as a Universal Serial Bus (USB) memory. The specific processing program 56 stored in a non-transitory storage medium is installed on the computer 22 of the data processing apparatus 12. The processor 28 executes specific processing in accordance with the specific processing program 56.

The specific processing program 56 may be stored in a storage device such as a server connected to the data processing apparatus 12 via the network 54, and the specific processing program 56 may be downloaded and installed on the computer 22 in response to a request from the data processing apparatus 12.

It is not necessary to store the entirety of the specific processing program 56 in a storage device such as a server connected to the data processing apparatus 12 via the network 54 or to store the entirety of the specific processing program 56 in the storage 32, and a part of the specific processing program 56 may be stored.

The following various processors may be used as hardware resources for executing the specific processing. Examples of the processor include a CPU which is a general-purpose processor functioning as a hardware resource that executes specific processing by executing a program, which is a software. Examples of the processor also include a dedicated electric circuit that is a processor having the circuit configuration specifically designed to execute specific processing, such as a Field-Programmable Gate Array (FPGA), a Programmable Logic Device (PLD), or an Application Specific Integrated Circuit (ASIC). A memory is built in or connected to any of these processors, and any of these processors execute the specific processing by using the memory.

The hardware resource that executes the specific processing may be configured by one of these various processors, or may be configured by a combination of two or more processors of the same type or different types (for example, a combination of plural FPGAs or a combination of a CPU and an FPGA). The hardware resource that executes the specific processing may be one processor.

As an example of the configuration including one processor, first, there is a configuration in which one processor is configured by a combination of one or more CPUs and software, and the processor functions as a hardware resource that executes the specific processing. Second, there is a configuration of using a processor that realizes a function of the entire system including plural hardware resources that execute the specific processing on one IC chip, such as represented by System-on-a-chip (SoC) or the like. As described above, the specific processing is realized by using one or more of the above-described various processors as hardware resources.

More specifically, an electric circuit in which circuit elements such as semiconductor elements are combined may be used as a hardware structure of these various processors. The above-described specific processing is merely an example. Thus, it is needless to say that unnecessary steps may be deleted, new steps may be added, or the processing order may be changed within a range not departing from the gist.

The contents described and illustrated above are detailed descriptions of parts according to the technique of the disclosure, and are merely examples of the technique of the disclosure. For example, the above description regarding the configuration, function, operation, and effect is a description regarding an example of the configuration, function, operation, and effect of the portion according to the technique of the disclosure. Therefore, it is needless to say that unnecessary portions may be deleted, new elements may be added, or replacement may be made with respect to the above described and illustrated contents without departing from the gist of the technique of the disclosure. Furthermore, in order to avoid complication and to facilitate understanding of the portion according to the technique of the disclosure, in the description content and the illustrated content described above, description regarding technical common sense or the like that does not require any particular description in enabling implementation of the technique of the disclosure is omitted.

All documents, patent applications, and technical standards described in this specification are incorporated herein by reference to the same extent as if each document, patent application, and technical standard were specifically and individually indicated to be incorporated by reference.

What is claimed is:

1. A data processing apparatus comprising:
a processor that is configured to:
acquire subject information indicating a daily state of a subject;
input a first prompt to a generation model that generates information according to input data, the first prompt being a prompt for outputting a characteristic of a developmental disorder and a degree of disorder of the subject indicated by the acquired subject information; and
output the characteristic of the developmental disorder and the degree of the disorder of the subject, which are output results of the generation model, to a predetermined output target.

2. The data processing apparatus according to claim 1, wherein the processor is further configured to:
add, to the first prompt, an instruction for outputting an observation regarding development of the subject indicated by the acquired subject information; and
output, to the predetermined output target, the characteristic of the developmental disorder and the degree of the disorder of the subject, which are the output results of the generation model, and the observation regarding the development of the subject.

3. The data processing apparatus according to claim 2, wherein the processor is further configured to:
acquire a user input regarding at least one of course consultation in an elementary school, course consultation in a middle school, course consultation in a high school, or course consultation after graduation from a high school of the subject;
add, to the first prompt, an instruction for outputting an answer to the course consultation indicated by the acquired user input; and
output, to the predetermined output target, the characteristic of the developmental disorder and the degree of the disorder of the subject, which are the output results of the generation model, the observation regarding the development of the subject, and the answer regarding the course consultation.

4. The data processing apparatus according to claim 1, wherein the processor is further configured to:
acquire actual age information indicating an actual age of the subject;
add, to the first prompt, an instruction for outputting a developmental age of the subject according to the actual age indicated by the acquired actual age information; and
output, to the predetermined output target, the characteristic of the developmental disorder and the degree of the disorder of the subject, which are the output results of the generation model, and the developmental age according to the actual age of the subject.

5. The data processing apparatus according to claim 4, wherein the processor is further configured to:
acquire future age information indicating a future age of the subject;
add, to the first prompt, an instruction for outputting a developmental age of the subject according to the future age indicated by the acquired future age information; and
output, to the predetermined output target, the characteristic of the developmental disorder and the degree of the disorder of the subject, which are the output results of the generation model, the developmental age according to the actual age of the subject, and the developmental age according to the future age of the subject.

6. The data processing apparatus according to claim 1, wherein the processor is further configured to:
acquire actual age information indicating an actual age of the subject;
input, to the generation model, a second prompt that is a prompt for outputting the daily state of the subject to be added by next information update time, according to the actual age indicated by the acquired actual age information, the characteristic of the developmental disorder and the degree of the disorder of the subject, which are output to the predetermined output target; and
output the daily state of the subject to be added, which is an output result of the generation model, to the predetermined output target at a timing at which a period until the next information update time is within a predetermined number of days.

7. The data processing apparatus according to claim 1, wherein the processor is further configured to:
acquire a user input regarding a trouble about the subject;
input, to the generation model, a third prompt that is a prompt for outputting an answer based on the characteristic of the developmental disorder of the subject, for the trouble indicated by the acquired user input; and
output, to the predetermined output target, an answer to the trouble about the subject, which is an output result of the generation model.

8. The data processing apparatus according to claim 1, wherein the processor is further configured to:
acquire a user input asking about a person or facility compatible with the subject;
input, to the generation model, a fourth prompt that is a prompt for outputting the person or facility compatible with the subject indicated by the acquired user input based on the characteristic of the developmental disorder and the degree of the disorder of the subject; and
output, to the predetermined output target, the person or facility compatible with the subject, which is an output result of the generation model.

9. A data processing method for causing a computer to execute a process comprising:
acquiring subject information indicating a daily state of a subject;
inputting a first prompt to a generation model that generates information according to input data, the first prompt being a prompt for outputting a characteristic of a developmental disorder and a degree of disorder of the subject indicated by the acquired subject information; and
outputting the characteristic of the developmental disorder and the degree of the disorder of the subject, which are output results of the generation model, to a predetermined output target.

10. A non-transitory storage medium storing a program for causing a computer to execute data processing, the data processing comprising:
acquiring subject information indicating a daily state of a subject;
inputting a first prompt to a generation model that generates information according to input data, the first prompt being a prompt for outputting a characteristic of a developmental disorder and a degree of disorder of the subject indicated by the acquired subject information; and
outputting the characteristic of the developmental disorder and the degree of the disorder of the subject, which are output results of the generation model, to a predetermined output target.
